(19) United States
(12) Patent Application Publication
(10) Pub. No.: US 2016/0243023 A1
(43) Pub. Date: Aug. 25, 2016

(54) Novel Skin Remodeling Strategy

(71) Applicant: ELC MANAGEMENT LLC, Melville, NY (US)

(72) Inventors: Ilse Sente, Zonhoven (BE); Hugo A.L. CORSTJENS, Maaseik (BE); Lieve DECLERCQ, Ekeren (BE)

(21) Appl. No.: 15/018,962

(22) Filed: Feb. 9, 2016

Related U.S. Application Data
(60) Provisional application No. 62/118,024, filed on Feb. 19, 2015.

(51) Int. Cl. A61K 8/99; A61K 8/97; G01N 33/50; A61K 8/98; A61Q 19/08; A61K 8/64; A61K 8/73

(52) U.S. Cl. CPC A61K 8/99; A61K 8/64; A61K 8/97; A61K 8/73; A61K 8/986; A61Q 19/08; G01N 33/5044; G01N 33/5023; G01N 2333/78

(57) ABSTRACT

The present invention relates to a method for identifying materials having an efficacy for improving collagen turnover in the skin. The invention also concerns a composition containing a combination of a collagen-degrading active and a collagen synthesis-stimulating active for enhancing skin remodeling.

### FIELD OF THE INVENTION

The present invention is concerned with the field of anti-aging of skin. More specifically, the invention relates to novel compositions and methods for preventing or eliminating lines and wrinkles in aging skin, and more particularly, lines and wrinkles in difficult to treat areas of the skin.

### BACKGROUND OF THE INVENTION

Aging, and photoaging, in particular, is associated with structural changes in skin which culminate in the appearance of fine lines, wrinkles, and sagging of the facial skin. Development of lines and wrinkles in the skin is a natural part of aging. Nevertheless, the presence of lines and wrinkles in the skin is a constant reminder of the loss of youth. Consumers' desire for the elimination of these undesirable signs of aging has led to a global business in anti-aging products and methods for rejuvenating the skin for a more youthful appearance.

Wrinkles are caused by a combination of factors, such as age, exposure to ultraviolet (UV) light, smoking, and repeated facial expressions. Decreased production of natural oils makes skin drier and appear to be more wrinkled. When skin loses moisture, the resulting dryness can lead to lines and wrinkles. Fat in the deeper layers of skin, which gives younger skin a plump appearance, starts to decline causing looser, sagging skin with pronounced lines and deeper folds. Exposure to UV light speeds up the natural aging process by facilitating a breakdown of the skin's connective tissue, consisting of collagen and elastin fibers, which lies beneath the epidermis in the deeper dermis of the skin. Without the supportive connective tissue, skin loses strength and flexibility. As a result, skin begins to sag and wrinkle prematurely. Smoking may also accelerate the normal aging process of skin, contributing to wrinkles. Repeated muscle movements from smiling, laughing and squinting may lead to the development of creases in the skin. Constant fluctuations in weight continually stretch the skin which makes wrinkling more likely. Each time a facial muscle is used, a groove forms beneath the surface of the skin. And as skin ages, it loses its flexibility and so is less able to spring back into place. As a result of this loss in elasticity, these grooves then become permanent features on the face in the form of deep wrinkles.

Treatments for preventing, retarding, arresting and/or reversing the development of lines and wrinkles in the skin range from cosmetics creams, containing various actives, such as retinoids and antioxidants, to resurfacing treatments, such as dermabrasion, microdermabrasion, laser, chemical peel, Botulinum toxin type A (Botox), soft tissue fillers, cosmetic surgery (face lift), and the use of devices which emit light of specified energies (e.g., near infra-red light).

The efficacy of commercially available non-prescription anti-wrinkle creams depends in part on the active ingredient or ingredients, However, as these creams contain lower concentrations of active ingredients than do prescription creams, claims and efficacy, if any, are limited and usually short-lived. Resurfacing treatments and cosmetic surgery may be associated with side effects, including redness, tenderness and pain. There is still a need for efficacious anti-aging products and methods which demonstrate minimal side effects.

### SUMMARY OF THE INVENTION

In one embodiment of a first aspect of the present invention, a method is provided for identifying a material having an efficacy for enhancing the activity of a collagen promoting active, comprising the steps of:

(a) incubating human dermal fibroblasts (HDFs) in the presence or absence of a test material;

(b) thereafter, removing the test material and incubating the HDFs in the presence of a collagen-promoting active for a time sufficient to stimulate collagen release by HDFs; and

(c) measuring an amount of collagen released by HDFs treated with the test material and with the collagen promoting active and comparing that amount with an amount of collagen released by HDFs treated with the collagen-promoting active in the absence of the test material.

In a further embodiment of this aspect of the invention, a method for identifying a material having an efficacy for enhancing the activity of a collagen synthesis-promoting active, comprises the steps of:

(a) incubating human dermal fibroblasts (HDFs) in the presence of a collagen synthesis-promoting active or in the presence of a combination of a collagen synthesis-promoting active with a test material for a time sufficient to stimulate collagen release by the HDFs; and

(b) measuring an amount of collagen released by HDFs treated with both the test material and the collagen synthesis-promoting active and comparing that amount with an amount of collagen released by HDFs treated with the collagen synthesis-promoting active in the absence of the treatment with the test material.

In a first embodiment of a second aspect of the invention, a method of enhancing collagen turnover in the skin is provided, the method comprising the steps of:

(a) applying to the skin in need of enhanced collagen turnover a first composition comprising a first active having an efficacy for degrading collagen in connective tissue in the skin and a cosmetically acceptable vehicle; and allowing the first composition to remain in contact with the skin for a time sufficient to stimulate collagen phagocytosis by fibroblasts in the connective tissue;

(b) applying to the skin a second composition comprising a second active having an efficacy for promoting collagen synthesis and a cosmetically acceptable vehicle; and allowing the second composition to remain in contact with the skin for a time sufficient to promote collagen release by fibroblasts in the connective tissue in the skin; wherein steps (a) and (b) occur sequentially in any order; and wherein the first active improves the activity of the second active when both the first composition and the second composition are applied to the skin.

A second embodiment of this aspect of the invention provides a method of enhancing collagen turnover in the skin, comprising the steps of:

(a) applying to the skin in need of enhanced collagen turnover a composition comprising a first active having an efficacy for degrading collagen by stimulating collagen phagocytosis by fibroblasts in connective tissue in the skin and a second active having an efficacy for promoting collagen synthesis by fibroblasts in the connective tissue and a cosmetically acceptable vehicle, wherein the first active improves the efficacy of the second active when the composition is applied to the skin; and

(b) permitting the composition to remain in contact with the skin for a time sufficient to enhance collagen turnover in the skin.

A third aspect of the present invention is concerned with a method for cosmetically improving human skin through a treatment regime for enhancing collagen turnover in the skin comprising:

providing a first composition containing a first active having an efficacy for degrading collagen in connective tissue in the skin and a cosmetically acceptable vehicle, the first composition functioning to stimulate collagen phagocytosis by fibroblasts in the connective tissue;

providing a second composition containing a second active having an efficacy for promoting collagen synthesis in connective tissue in the skin and a cosmetically acceptable vehicle, the second composition functioning to promote collagen release by fibroblasts in the connective tissue;

instructing consumers on the use of the first and second compositions in a sequential manner to achieve a level of collagen turnover in the skin which exceeds a level of collagen turnover achievable by treatment with the second composition in the absence of treatment with the first composition;

applying the first composition to the skin; and applying the second composition to the skin; wherein the first composition and the second composition may be applied to the skin in any order; and wherein the first active improves the efficacy of the second active when both the first composition and the second composition are applied to the skin.

In a further embodiment of the third aspect of the invention, the method for cosmetically improving human skin through a treatment for enhancing collagen turnover in the skin, comprising:

(a) providing a composition comprising a first active having an efficacy for degrading collagen in connective tissue in the skin and a second active having an efficacy for promoting collagen synthesis by fibroblasts in the connective tissue and a cosmetically acceptable vehicle; wherein the first active improves the efficacy of the second active when the composition is applied to the skin; and

(b) applying the composition to the skin and retaining the composition in contact with the skin for a time sufficient for the first active to stimulate collagen phagocytosis by the fibroblasts in the connective tissue and for the second active to promote collagen release by the fibroblasts in the connective tissue.

A fourth aspect of the invention provides a skin treatment product comprising: a composition comprising a first active having an efficacy for degrading collagen by stimulating collagen phagocytosis by fibroblasts in connective tissue in the skin and a second active having an efficacy for promoting collagen synthesis by fibroblasts in the connective tissue and a cosmetically acceptable vehicle, wherein the first active and the second active are present in the composition in amounts effective for the first active to improve the efficacy of the second active when the composition is applied to the skin.

A fifth aspect of the present invention concerns a skin treatment kit. The kit comprises:

a first composition containing a first active having an efficacy for degrading collagen by stimulating collagen phagocytosis by fibroblasts in connective tissue in the skin and a cosmetically acceptable vehicle;

a second composition containing a second active having an efficacy for promoting collagen synthesis by fibroblasts in connective tissue in the skin and a cosmetically acceptable vehicle, wherein the first active improves the efficacy of the second active when both the first composition and the second composition are applied to the skin; and

instructions for consumers on the use of the first and second compositions in a sequential manner in any order to achieve a degree of collagen release which exceeds a degree of collagen release achievable by treatment with the second composition in the absence of treatment with the first composition.

### BRIEF DESCRIPTION OF THE DRAWINGS

FIG. 1 is a graph showing the effect of Wild Bush Plum extract on Transforming Growth Factor Beta 1 (TGFβ1)-induced collagen levels in HDFs.

FIG. 2 is a graph showing the effect of Pichia/resveratrol ferment on TGFβ1-induced collagen levels in HDFs.

FIG. 3 is a graph showing the effect of Neovixyl (rice stem cell and grape seed extracts) on TGFβ1-induced collagen levels in HDFs.

FIG. 4 is a graph showing the effect of Amacha liquid B (Hydrangea macrohylla var. Oamacha Makino) plant extract on TGFβ1-induced collagen levels in HDFs.

FIG. 5 is a graph showing the effect of Theobroma (cacao seed extract) on TGFβ1-induced collagen levels in HDFs.

FIG. 6 is a graph showing the effect of Bifidus extract (Lactobacillus Bifidus) on TGFβ1-induced collagen levels in HDFs.

FIG. 7 is a graph showing the effect of Taisoh liquid (Zizphus jujube date extract) on TGFβ1-induced collagen levels in HDFs.

FIG. 8 is a graph showing the effect of NXP75 (protein concentrate fraction from pasteurized fluid whey) on TGFβ1-induced collagen levels in HDFs.

FIG. 9 is a graph showing the effect of NXP75 on Solpeptide (Solanum tuberosum) induced collagen levels in HDFs.

FIG. 10 is a graph showing the effect of methylglyoxal on collagen-coated microsphere beads.

FIG. 11 is a graph showing the effect on phagocytosis by HDFs of collagen-coated microsphere beads pretreated with methylglyoxal.

FIG. 12 is a graph showing the effect of Neovixyl on collagen synthesis stimulated by NXP75.

FIG. 13 is a graph showing the effect of Amacha liquid B on collagen synthesis stimulated by NXP75.

FIG. 14 is a graph showing the effect of Taisoh liquid on collagen synthesis induced by Mitostime.

FIG. 15 is a graph showing the effect of Taisoh liquid on collagen synthesis induced by Solpeptide.

FIG. 16 is a graph showing the effect of Taisoh liquid on collagen synthesis induced by an extract of the yeast Hansenula polymorpha.

FIG. 17 is a graph showing the effect of Taisoh liquid on collagen synthesis induced by Riboxyl.

FIG. 18 is a graph showing the effect of Amacha liquid B on collagen synthesis induced by an extract of the yeast Hansenula polymorpha.

### DETAILED DESCRIPTION OF THE INVENTION

Human dermal fibroblasts (HDFs) generate connective tissue to unite separate cells layers. HDFs produce protein molecules, including fibronectin, collagen, and elastin, which form the extracellular matrix. Long, fibrous bands of collagen (i.e., fascia) anchor skin to the muscles and organs of the body. By creating extracellular matrix between dermis and epidermis, fibroblasts allow epithelial cells to affix the matrix allowing epidermal cells to join together to form the top layer of skin (i.e., organized squamous epithelial cells).

Collagen, the main protein of connective tissue, is responsible for skin strength and elasticity. With age, the human body loses the ability to rebuild collagen. In soft connective tissue, homeostasis is maintained by fibroblasts that both synthesize and degrade collagenous matrix in response to growth and development and also pathological conditions. Disruptions to the balance of matrix remodeling may lead to net loss of collagen or to disorganized overgrowth of collagen. Two pathways of collagen turnover have been recognized, an intracellular one and an extracellular one. The extracellular pathway is thought to depend upon the activity of proteinases, and in particular, metalloproteinases (MMPs), released adjacent collagen fibrils by HDFs, while the intracellular route is believed to involve phagocytosis of collagen fibrils by HDFs. Phagocytosis is followed by digestion of the collagen fibrils in the lysosomal apparatus by proteinases.

As we age, however, age-related changes are unavoidable, and fibroblasts, which are responsible for maintaining the collagen homeostasis, are less able to keep up. This is most often apparent in the periorbital regions of the skin. The skin around the eyes is the thinnest in the body, containing little collagen and elastin. Upon acquiring sun damage, this skin is among the first areas on the face to wrinkle, resulting in “crow's feet”. These areas, characterized by stiffened, aligned collagen fibrils, are particularly resistant to the natural homeostatic mechanism of collagen turnover which coordinates synthesis and degradation of collagen.

It is thought that, when unwrinkled skin is flexed to form a temporary expression fold, the greatest tension is experienced by the tissue at the base of the fold. In response to that tension, fibroblasts deposit collagen in the direction of the tension or perpendicular to the fold. Each successive time this skin is flexed and the fold forms, the tissue at the base of the fold is just a little stiffer due to the deposited collagen. That makes the tension at the wrinkle base just a little higher and induces the deposition of still more aligned collagen. Over time, the aligned collagen comes to dominate the stress field in the skin to the point where it maintains a permanent wrinkle.

As crow's feet in the periorbital areas are characterized by the presence of stiffened, aligned collagen which is resistant to the extracellular route of collagen degradation by MMPs, the inventors investigated whether the physiological remodeling of connective tissue could be boosted by stimulating the intracellular route of collagen degradation (i.e., phagocytosis of collagen by fibroblasts) in combination with the use of collagen promoters.

The inventors have developed new clinical remodeling strategies to identify new actives which boost the effect of promoters of collagen synthesis. A first strategy utilizes a two-step approach. In step 1, HDFs are treated with a collagen phagocytosis stimulating active, while in step 2, the HDFs are treated with a collagen promoting active.

The method for identifying a material having an efficacy for enhancing the activity of a collagen promoting active, comprises the steps of:

(a) incubating HDFs in the presence or absence of a test material;

(b) thereafter, removing the test material and incubating the HDFs in the presence of a collagen-promoting active for a time sufficient to stimulate collagen release by HDFs; and

(c) measuring an amount of collagen released by HDFs treated with the test material and with the collagen promoting active and comparing that amount with an amount of collagen released by HDFs treated with the collagen-promoting active in the absence of the test material.

Preferably, in step (a), the HDFs are incubated in the presence or absence of the test material for about 48 hours, and in step (b), the HDFs are incubated in the presence of the collagen synthesis-promoting active for about 72 hours, according to standard procedure for the in vitro evaluation of collagen enhancers on collagen production by HDFs. Measuring the amount of collagen released in step (c) is carried out using a Procollagen Type 1 C-Peptide Enzyme-linked immunosorbent assay (PIP EIA).

In a second strategy, a method for identifying a material having an efficacy for enhancing the activity of a collagen synthesis-promoting active, comprises the steps of:

(a) incubating HDFs in the presence of a collagen synthesis-promoting active or in the presence of a combination of a collagen synthesis-promoting active with a test material for a time sufficient to stimulate collagen release by the HDFs; and

(b) measuring an amount of collagen released by HDFs treated with both the test material and the collagen synthesis-promoting active and comparing that amount with an amount of collagen released by HDFs treated with the collagen synthesis-promoting active in the absence of the treatment with the test material.

Measuring the amount of collagen released in step (b) is carried out using the PIP EIA assay.

The present invention further concerns a method of enhancing collagen turnover in the skin. In a first embodiment of the invention, the method includes the steps of:

(a) applying to the skin in need of enhanced collagen turnover a first composition comprising a first active having an efficacy for degrading collagen in connective tissue in the skin and a cosmetically acceptable vehicle; and allowing the first composition to remain in contact with the skin for a time sufficient to stimulate collagen phagocytosis by fibroblasts in the connective tissue;

(b) applying to the skin a second composition comprising a second active having an efficacy for promoting collagen synthesis and a cosmetically acceptable vehicle; and allowing the second composition to remain in contact with the skin for a time sufficient to promote collagen release by fibroblasts in the connective tissue in the skin; wherein steps (a) and (b) occur sequentially in any order; and wherein the first active improves the activity of the second active when both the first composition and the second composition are applied to the skin.

In a second embodiment of this aspect of the invention, the method of enhancing collagen turnover in the skin comprises the steps of:

(a) applying to the skin in need of enhanced collagen turnover a composition comprising a first active having an efficacy for degrading collagen by stimulating collagen phagocytosis by fibroblasts in connective tissue in the skin and a second active having an efficacy for promoting collagen synthesis by fibroblasts in the connective tissue and a cosmetically acceptable vehicle, wherein the first active improves the efficacy of the second active when the composition is applied to the skin; and (b) permitting the composition to remain in contact with the skin for a time sufficient to enhance collagen turnover in the skin.

The present invention also is concerned with a method for cosmetically improving human skin through a treatment for enhancing collagen turnover in the skin. In a first embodiment of this aspect of the invention, the method includes the steps of:

providing a first composition containing a first active having an efficacy for degrading collagen in connective tissue in the skin and a cosmetically acceptable vehicle, the first composition functioning to stimulate collagen phagocytosis by fibroblasts in the connective tissue;

providing a second composition containing a second active having an efficacy for promoting collagen synthesis in connective tissue in the skin and a cosmetically acceptable vehicle, the second composition functioning to promote collagen release by fibroblasts in the connective tissue;

instructing consumers on the use of the first and second compositions in a sequential manner to achieve a level of collagen turnover in the skin which exceeds a level of collagen turnover achievable by treatment with the second composition in the absence of treatment with the first composition;

applying the first composition to the skin; and

applying the second composition to the skin; wherein the first composition and the second composition may be applied to the skin in any order; and wherein the first active improves the efficacy of the second active when both the first composition and the second composition are applied to the skin.

In a second embodiment of this aspect of the invention, a method for cosmetically improving human skin through a treatment for enhancing collagen turnover in the skin comprises the steps of:

(a) providing a composition comprising a first active having an efficacy for degrading collagen in connective tissue in the skin and a second active having an efficacy for promoting collagen synthesis by fibroblasts in the connective tissue and a cosmetically acceptable vehicle; wherein the first active improves the efficacy of the second active when the composition is applied to the skin; and

(b) applying the composition to the skin and retaining the composition in contact with the skin for a time sufficient for the first active to stimulate collagen phagocytosis by the fibroblasts in the connective tissue and for the second active to promote collagen release by the fibroblasts in the connective tissue.

A skin treatment product according to the present invention comprises a composition comprising a first active having an efficacy for degrading collagen by stimulating collagen phagocytosis by fibroblasts in connective tissue in the skin and a second active having an efficacy for promoting collagen synthesis by fibroblasts in the connective tissue and a cosmetically acceptable vehicle, wherein the first active and the second active are present in the composition in amounts effective for the first active to improve the efficacy of the second active when the composition is applied to the skin.

A skin treatment kit according to the present invention includes:

a first composition containing a first active having an efficacy for degrading collagen by stimulating collagen phagocytosis by fibroblasts in connective tissue in the skin and a cosmetically acceptable vehicle;

a second composition containing a second active having an efficacy for promoting collagen synthesis by fibroblasts in connective tissue in the skin and a cosmetically acceptable vehicle, wherein the first active improves the efficacy of the second active when both the first composition and the second composition are applied to the skin; and

instructions for consumers on the use of the first and second compositions sequentially in any order to achieve a degree of collagen release which exceeds a degree of collagen release achievable by treatment with the second composition in the absence of treatment with the first composition.

Materials having an efficacy for promoting collagen synthesis which are useful in the products and the methods of the invention may include, but are not limited to, TGFβ1, Solpeptide, Calophyllum Inophyllum (available as Tamanu Original Oil), Palmitoyl Dipeptide-5 diaminobutyroyl hydroxythreonine/Palmitoyl Dipeptide-5 diaminohydroxybutyrate (Syn® Tacks 801045), Whey protein, Mimosa tenuiflora (available as Tepescohuite spray dried extract), Soybean polypetide, Rhodiola Rosea, Soybean extract (available as Phytomatrix K100), Palmitoyl Tripeptide-1/Palmitoyl Tripeptide-7, Algae extract/hydrolyzed Rice protein, Hippophae Rhamnoides (available as Sea Buckthorn extract), Vitis vinifera (available as Neovixyl), Opuntia Tuna (prickly pear cactus), Whey protein (available as NXP75), extract of the yeast Hansenula polymorpha (H. polymorpha), Palmitoyl Pentapeptide-4 (available as Matrixyl 500), Yeast polysaccharides (available as Metabiotics™ Microlift), Laminaria Digitata (available as Mitostime DI), Hydrolyzed collagen, Algae extract (available as Lanablue paraben free), Galactoarabinan (available as TRIspire Enhance), Laurydone, Terminalia Ferdinandiana (Kakadu Plum Extract (Glycerin)), Aminopropyl ascorbyl phosphate, Artemia, Psidium Guajava (Guava Extract), Zea mays, Crithmum Maritimum, Eryngium Maritimum, Creatine (available as Creapure®), Centella asiatica, Persea Gratissima (avocado fruit extract, available as Avocadin™ HU25), Scutellaria Baicalensis (available as Baicalin MM), Palmitoyl hexapeptide-12, Tetrahexyldecyl ascorbate (available as BV-OSC), Pseudoalteromonas ferment (available as Antarcticine®), Acetyl hexapeptide-8 (available as Argireline® solution), ascorbyl glucoside, Magnesium ascorbyl phosphate, and Ascorbyl palmitate.

Materials having an efficacy for degrading collagen which are useful in the products and the methods of the invention may include, but are not limited to, Neovixyl, Theobroma, NXP75, Taisoh liquid, Pichia/resveratrol ferment, Wild Bush Plum extract, Amacha liquid B, and Bifidus extract.

Skin treatments products according to the present invention may comprise one or two topical compositions. In the case of a single formula containing both a collagen-promoting active and a collagen-degrading active which improves the efficacy of the collagen-promoting active, this may take the form of, for example, a toner, a spritzer, a lotion, a day and/or a night cream, a day serum and/or a night repair serum, a mousse, a gel, as mask, a makeup foundation, and so forth. Alternatively, the skin may be treated to a regimen involving the application of two separate compositions, one of which contains a collagen-promoting active and the other of which contains a collagen-degrading active which improves the efficacy of the collagen-promoting active when both compositions are applied to the skin. Each of the two separate compositions may be provided, for example, in any one or more of the forms mentioned above with regard to the single formula.

The compositions may be applied to any skin areas which have developed lines and/or wrinkles, or any skin areas which are susceptible to the adverse effects of the environment, daily stress, sun exposure, or premature aging, and which may be expected to develop lines and/or wrinkles. The compositions of the present invention are particularly suited to address those skin areas which are most resistant to skin remodeling, such as the crow's feet in the skin of the periorbital regions around the eyes.

The compositions of the present invention may be applied to the skin on an as-needed basis or according to a pre-set schedule. The compositions may be applied directly to clean skin, before application of any other treatment product, or foundation makeup, or they may be applied over the other treatment product or foundation makeup. The amount of the composition(s) applied to the skin with each application can vary widely depending on the specific need of the user. For example, if the user has prominent wrinkles, the user may choose to apply the compositions more frequently than if the user's skin exhibits finer lines. The composition(s) may be applied for a period of days to months or even years, and at a frequency ranging from about once or twice a day to once a week. In another example, the single composition may be applied one or twice a day, morning and/or evening, for a period of six months or more. In a further example, where two compositions are to be applied sequentially, in a regimen, these may be used in any order. One composition may be applied to the skin immediately after the other composition is applied to the skin, one or two times per day. In another example, the separate compositions may applied once or twice a day, on alternate days, or the separate compositions may be applied one or two times a day during alternate weeks, and so forth.

The invention will be further described by the following non-limiting examples which are provided for the purposes of illustration only.

### EXAMPLES

### Example 1

### Measurement of TGFβ1-Induced Collagen Synthesis in HDF After Pretreatment with Inducers of Collagen Phagocytosis

### Procedure:

1. On day 1, HDFs were plated in 96 well plates.
2. On day 2, HDFs, after reaching confluence, were treated with medium (control) or with actives (test materials previously found by the inventors to be phagocytosis enhancers*; data not shown) under starvation conditions for 48 hours (step 1).
3. On day 4, the actives were removed and the cells were treated with medium or with a collagen inducer, 0.25 ng/ml TGFβ1 in medium, for 72 hours (step 2).
4. On day 7, medium was collected and analyzed for collagen release using a PIP EIA. The amount of PIP (pro-collagen ng/ml medium) is quantitated by measuring the absorbance using an EIA plate reader. Accurate sample concentrations of PIP can be determined by comparing the specific absorbances to a standard curve. Cell viability also was assessed using a standard 3-(4,5-dimethylthiazol-2-yl)-2,5-diphenyltetrazoliumbromide (MTT) assay. The assay involves the conversion of the water soluble MTT, a yellow tetrazole, to an insoluble purple formazan in living cells. The formazan is then solubilized and the concentration determined by measuring the absorbance at a wavelength of from about 500-600 nm using a spectrophotometer.

### *General Protocol for Measuring Efficacy of Active to Boost Phagocytosis:

The analysis of collagen phagocytosis by HDFs was evaluated using collagen-coated fluorescent microspheres (available from Molecular Probes as F20892 FluoSpheres® collagen I-labelled microspheres, 1.0 μm yellow-green fluorescent 500/515 nm beads) which attach to the cell surface and become engulfed. HDFs were treated with an active under starvation conditions for 48 hours and then incubated with collagen-coated microspheres (2×10⁶ beads) for 3 hours. The HDFs were then washed to remove loose or unbound microspheres. Fluorescence microscopy was used to confirm that the fluorescent microspheres were engulfed by HDFs. The fluorescent signal, read by a spectrophotometer, was used to quantitatively measure the collagen uptake by phagocytosis. The uptake by HDFs was found to be specific for the collagen coating as uncoated spheres were not retained by the cells. Each of the actives tested was found to boost phagocytosis of the collagen-coated microspheres compared with medium.

### Phagocytosis Enhancing Actives Used in Step 1:

1. Wild bush plum extract

2. Metabiotics

3. Neovixyl

4. Amacha liquid B plant extract

5. Theobroma

6. Bifidus extract

7. Taisoh liquid

8. NXP-75

### Results:

For all results shown in the graphs, the following calculation was employed to ascertain the percent increase in collagen, measured as procollagen (Pro-Col), production resulting from sequential treatment of HDFs with a test material followed by the collagen booster, TGFβ1, as compared with that amount of collagen produced using TGFβ1 in the absence of the test material: {[[amount of Pro-Col observed after treatment with test material in medium (step 1) and then with TGFβ1 in medium (step 2)]—[amount of Pro-Col observed after treatment with medium (step 1) and then with TGFβ1 in medium (step 2)]]/[[amount of Pro-Col observed after treatment with test material in medium (step 1) and then with medium (step 2)]—[amount of Pro-Col observed after treatment with medium (step 1) and then with medium (step 2)]]×100}−(100).

The statistical analysis method used on the data was ANOVA and Fisher LSD post hoc where *=p<0.05; **=p<0.01.

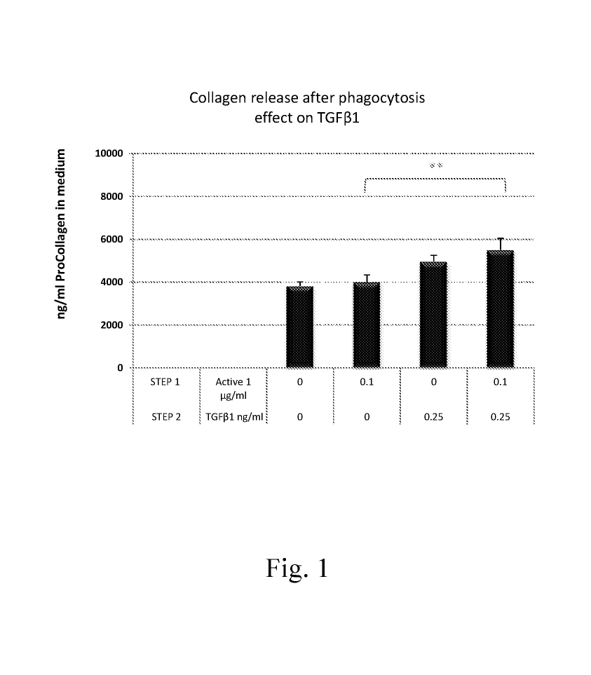

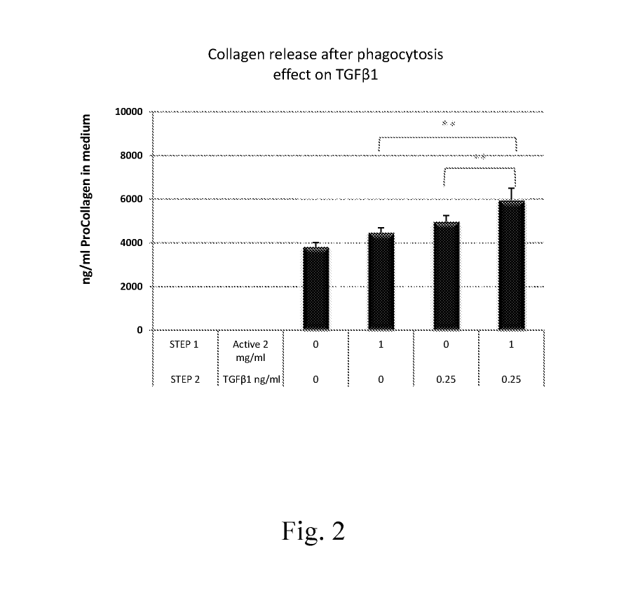

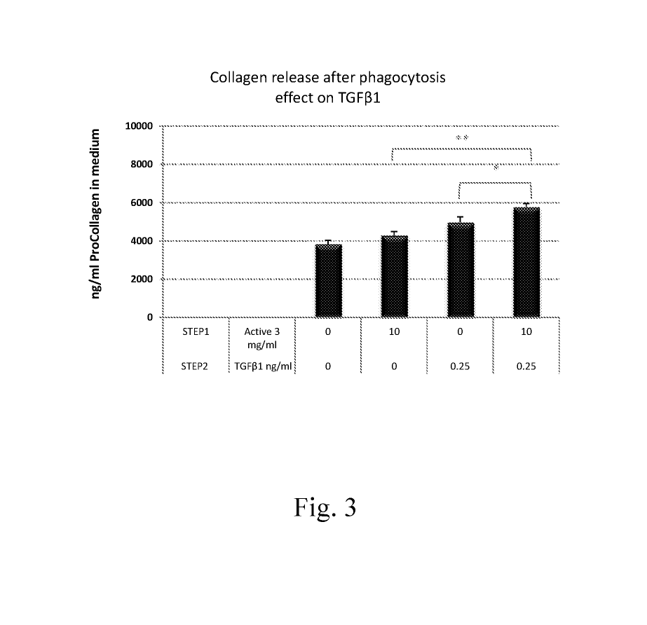

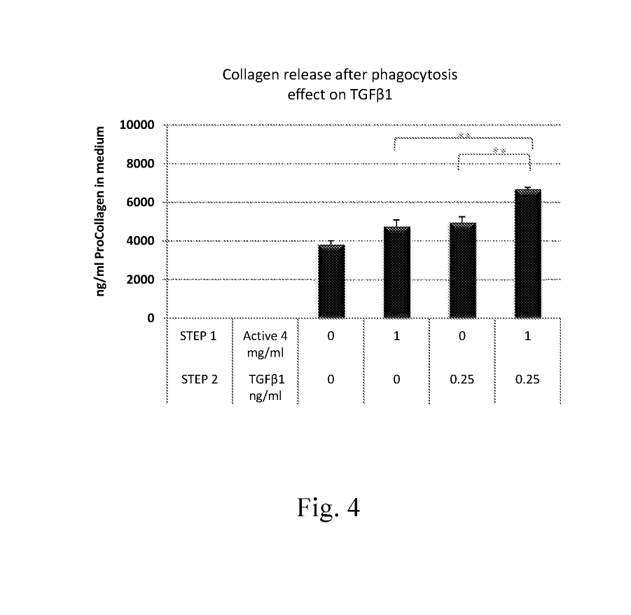

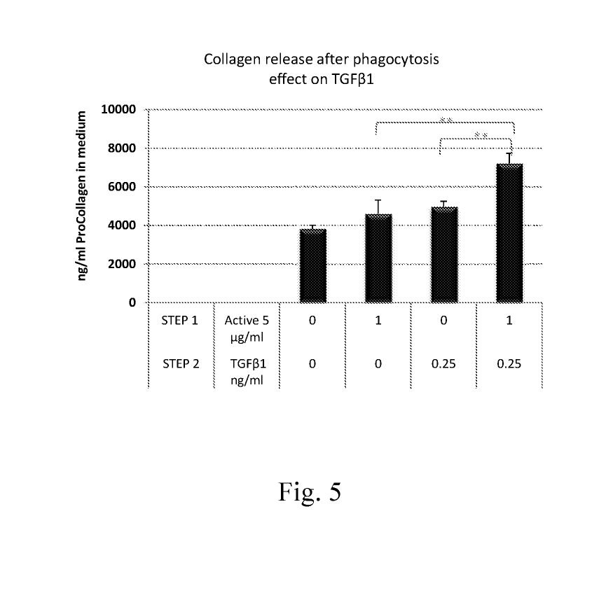

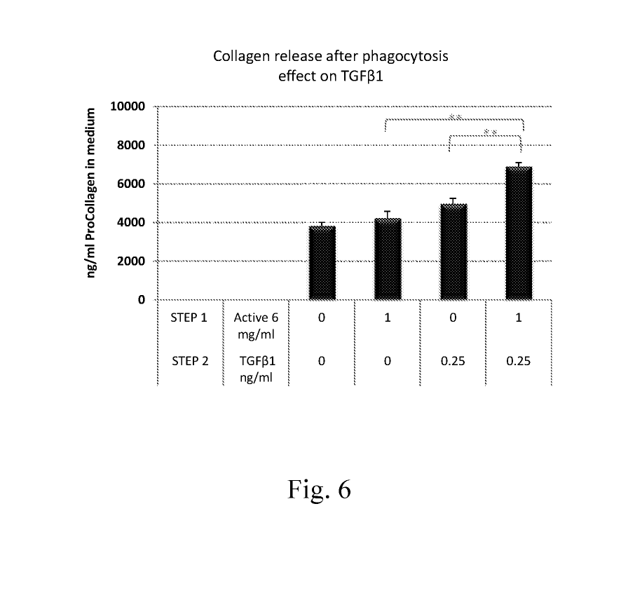

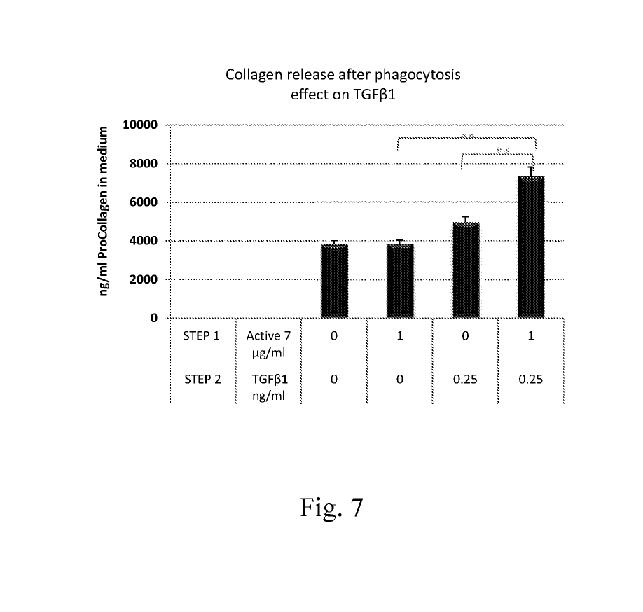

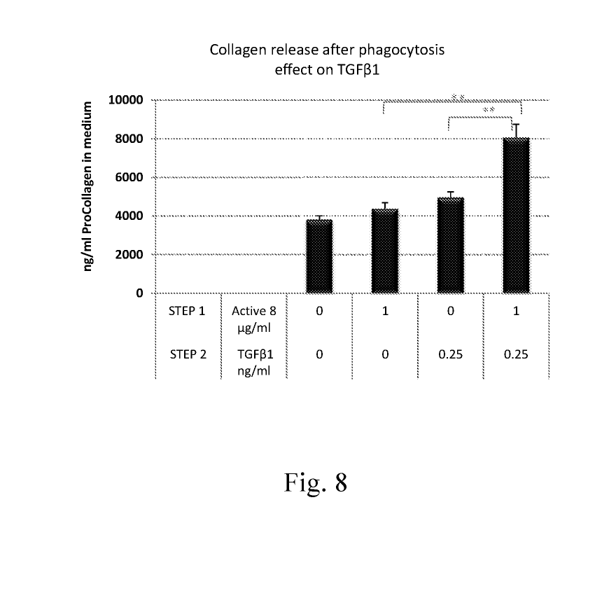

In each of FIGS. 1-8, the effect of each of the phagocytosis enhancing actives on the collagen synthesis stimulated by TGFβ1 is shown. TGFβ1, used alone at 0.25 ng/ml, was found to increase collagen synthesis by 30% compared with the non-treated control. In FIG. 1, it is demonstrated that 0.1 μg/m1 Wild Bush Plum extract increased the TGFβ1-induced collagen level by 187%. The effect was not statistically significant. The effect of Metabiotics lift, used at 1 mg/ml, was found to increase the level of TGFβ1-induced collagen by 51%, as shown in FIG. 2. This result is statistically significant. In FIG. 3, the effect of Neovixyl, used at 10 mg/ml, was found to increase the TGFβ1-induced collagen level by 78%. This effect also is statistically significant. Amacha liquid B plant extract, used at 1 mg/ml, was demonstrated to increase the amount of collagen induced by TGFβ1 by a statistically significant 85%, as seen in FIG. 4. Theobroma, tested at 1 μug/ml, was found to increase the TGFβ1-induced collagen level by a statistically significant 187%, as shown in FIG. 5. The results shown in FIG. 6 indicate that bacterial broth Bifidus, used at 1 mg/ml, increased the TGFβ1-induced collagen level by a statistically significant 376%. Taisoh liquid, tested at 1 μg/ml, was found to increase the level of TGFβ1-induced collagen by a statistically significant 5373%, as shown in FIG. 7. The results shown in FIG. 8, demonstrate that NXP75, tested at 1 μg/ml, increased the level of TGFβ1-induced collagen by a statistically significant 450%.

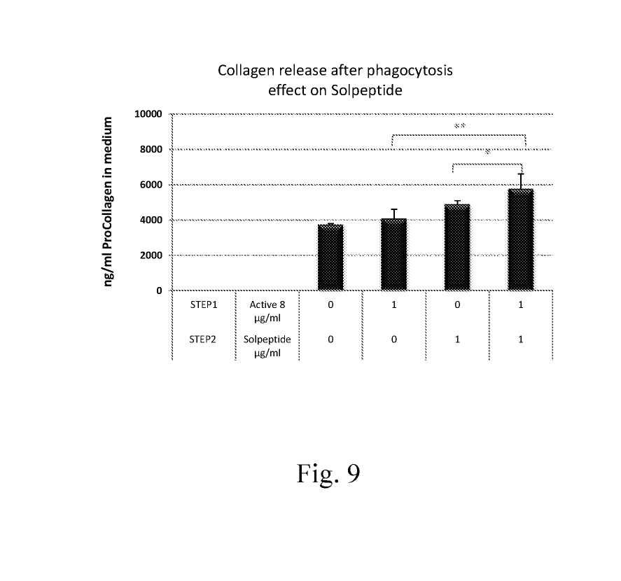

The results shown in FIG. 9 demonstrate that NXP75, tested at 1 μg/ml, also increased the collagen synthesis induced by a different collagen synthesis enhancer, Solpeptide, by a statistically significant 166%.

### Example 2

### Measurement of NXP75-Induced Collagen Synthesis in HDF After Pretreatment with Inducers of Collagen Phagocytosis

### Procedure:

1. On day 1, HDFs were plated in 96 well plates.
2. On day 2, HDFs, after reaching confluence, were treated with medium (control) or with actives under starvation conditions for 48 hours (step 1). Actives were test materials previously found by the inventors to be phagocytosis enhancers; data not shown (see Example 1 above for General Protocol for Measuring Efficacy of Active to Boost Phagocytosis).
3. On day 4, the actives were removed and the cells were treated with medium or with a collagen inducer, 20 μg/ml NXP75 in medium, for 72 hours (step 2).
4. On day 7, medium was collected and analyzed for collagen release using a PIP EIA, as described in Example 1, hereinabove.

### Results:

For all results shown in the graphs, the following calculation was employed to ascertain the percent increase in collagen, measured as procollagen (Pro-Col), production resulting from sequential treatment of HDFs with a test material followed by the collagen booster, NXP75, as compared with that amount of collagen produced using NXP75 in the absence of the test material: {[[amount of Pro-Col observed after treatment with test material in medium (step 1) and then with NXP75 in medium (step 2)]−[amount of Pro-Col observed after treatment with medium (step 1) and then with NXP75 in medium (step 2)]]/[[amount of Pro-Col observed after treatment with test material in medium (step 1) and then with medium (step 2)]−[amount of Pro-Col observed after treatment with medium (step 1) and then with medium (step 2)]]×100}−(100).

The statistical analysis method used on the data was ANOVA and Fisher LSD post hoc where *=p<0.05; **=p<0.01.

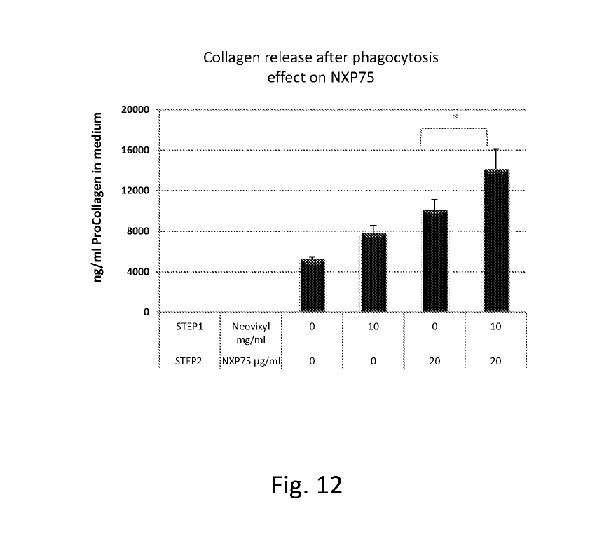

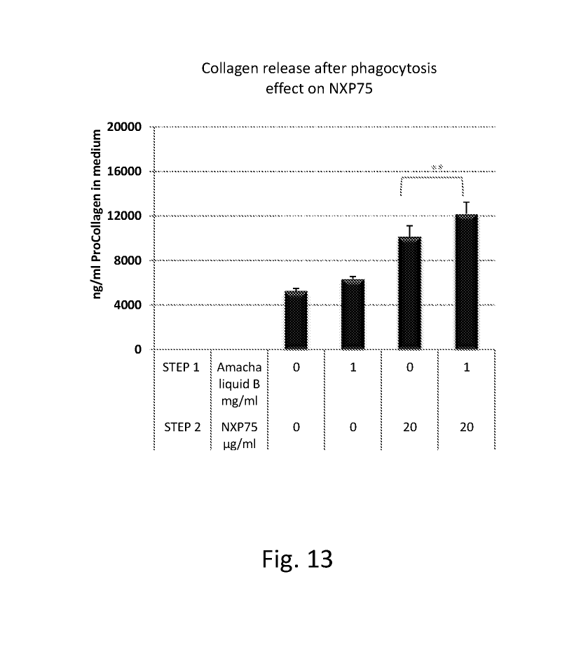

In each of FIGS. 12 and 13, the effect of each of the phagocytosis enhancing actives on the collagen synthesis stimulated by NXP75 is shown. In FIG. 12, it is shown that Neovixyl, used at 10 mg/ml, increased the NXP75-induced collagen synthesis by a statistically significant 55%. Phagocytosis enhancer Amacha liquid B, used at 1 mg/ml, was observed to increase the NXP75-induced collagen synthesis by a statistically significant 94%.

### Example 3

### Measurement of NXP75-Induced Collagen Synthesis in HDF After Pretreatment with Inducers of Collagen Phagocytosis

### Procedure:

1. On day 1, HDFs were plated in 96 well plates.
2. On day 2, HDFs, after reaching confluence, were treated with medium (control) or with actives under starvation conditions for 48 hours (step 1). Actives were test materials previously found by the inventors to be phagocytosis enhancers; data not shown (see Example 1 above for General Protocol for Measuring Efficacy of Active to Boost Phagocytosis).
3. On day 4, the actives were removed and the cells were treated with medium or with a collagen inducer, 20 μg/ml NXP75 in medium, for 72 hours (step 2).
4. On day 7, medium was collected and analyzed for collagen release using a PIP EIA, as described in Example 1, hereinabove.

### Results:

For all results shown in the graphs, the following calculation was employed to ascertain the percent increase in collagen, measured as procollagen (Pro-Col), production resulting from sequential treatment of HDFs with a test material followed by the collagen booster, NXP75, as compared with that amount of collagen produced using NXP75 in the absence of the test material:

{[[amount of Pro-Col observed after treatment with test material in medium (step 1) and then with NXP75 in medium (step 2)]−[amount of Pro-Col observed after treatment with medium (step 1) and then with NXP75 in medium (step 2)]]/[[amount of Pro-Col observed after treatment with test material in medium (step 1) and then with medium (step 2)]−[amount of Pro-Col observed after treatment with medium (step 1) and then with medium (step 2)]]×100}−(100).

The statistical analysis method used on the data was ANOVA and Fisher LSD post hoc where *=p<0.05; **=p<0.01.

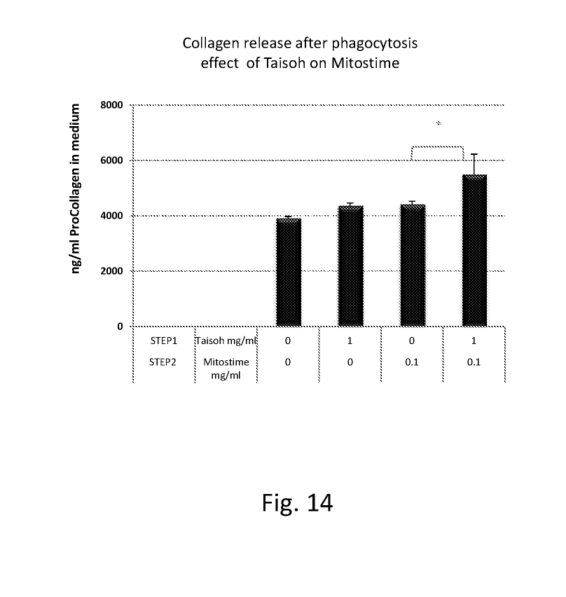

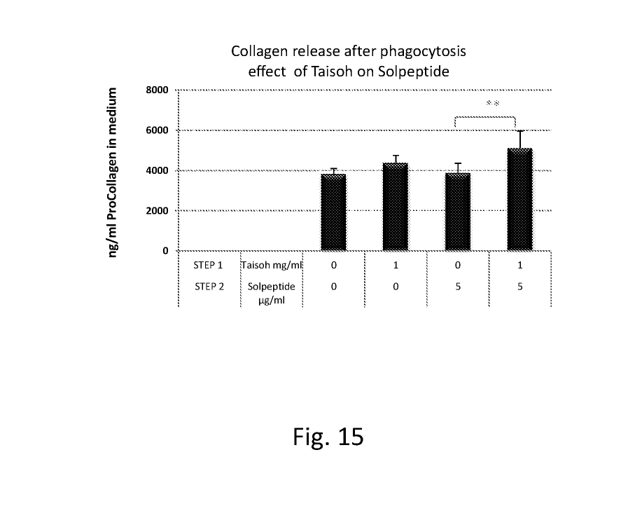

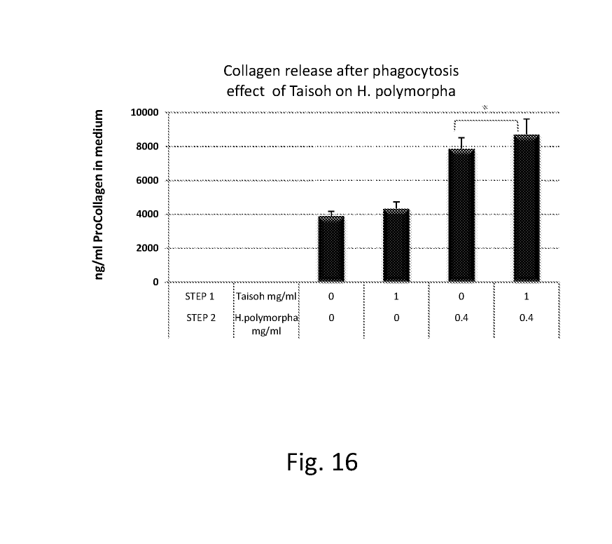

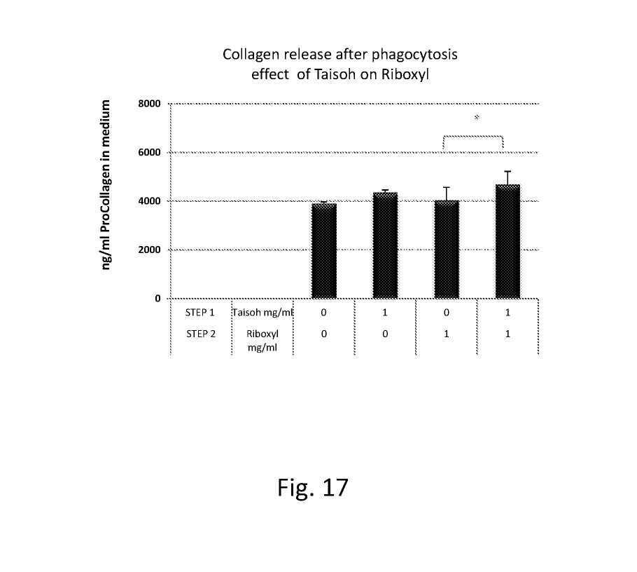

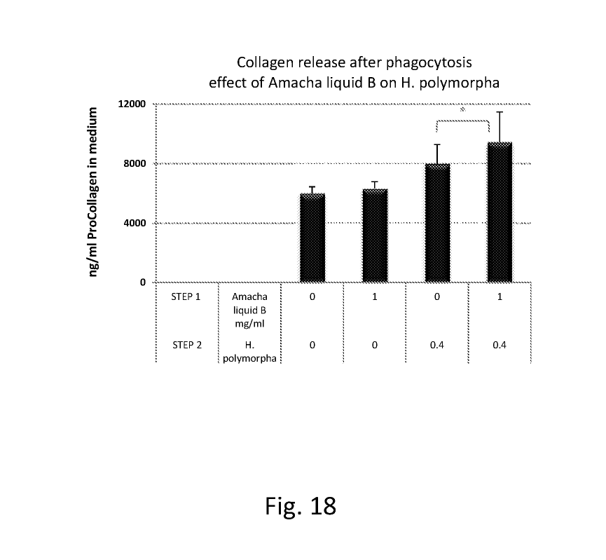

In each of FIGS. 14-18, the effect of each of the phagocytosis enhancing actives on the collagen synthesis stimulated by collagen-boosting actives is shown. In FIG. 14, phagocytosis enhancer Taisoh liquid, used at 1 mg/ml, was found to boost collagen synthesis induced by Mitostime used at 0.1 mg/ml. The mitostime-induced collagen synthesis was boosted by a statistically significant 141%. Taisoh liquid, used at lmg/ml, also was observed to boost the collagen synthesis induced by Solpeptide used at 5 μg/ml. As shown in FIG. 15, collagen synthesis was boosted by a statistically significant 125%. Taisoh liquid, used at 1 mg/ml, was further shown to boost the collagen synthesis induced by an extract of the yeast Hansenula polymorpha, used at 0.4 mg/ml. As shown in FIG. 16, collagen synthesis was boosted by a statistically significant 87%. As is also shown in FIG. 17, Taisoh liquid, used at 1 mg/ml boosts the collagen synthesis induced by Riboxyl, used at 1 mg/ml by a statistically significant 49%. Phagocytosis enhancer, Amacha B liquid B, used at 1 mg/ml was observed to increase the collagen synthesis induced by an extract of the yeast Hansenula polymorpha, used at 0.4 mg/ml. by a statistically significant 348%, as observed in FIG. 18.

### Example 4

### Effect of Glycation of the Collagen on Collagen Phagocytosis by HDFs

Advanced glycation end product (AGE)-modified collagen is characterized by cross-links causing stiffness and reduced elasticity due to a decreased susceptibility to proteolytic degradation, such as by MMPs (i.e., the extracellular pathway of collagen turnover). As skin areas, such as crow's feet in the periorbital regions, are characterized by stiffened collagen that, similarly, is resistant to degradation by MMPs, the inventors investigated how glycation may affect phagocytosis of collagen by HDFs.

### A. Exposure of Collagen-Coated Microspheres to Methylglyoxal

### Procedure:

1. Collagen-coated microspheres were incubated overnight with glycation inducer, methylglyoxal.
2. The microspheres were then incubated with an amine reactive fluorescent dye (Alexa Fluor 594 NHS ester) and the dye's fluorescence was measured.

The impact of the oxidation reaction between methylglyoxal and collagen was measured by probing for the amount of free, unmodified amine groups on collagen (the targets for the glycation reaction with methylglyoxal). A lower reaction rate with the probe is indicative of the presence of glycated collagen.

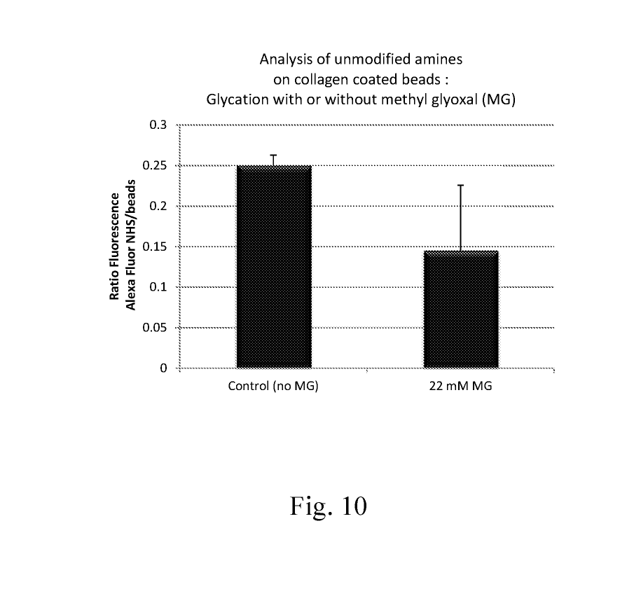

Results, shown in FIG. 10, indicate that the fluorescent signal for free, unmodified amine functionalities of the collagen is lower for collagen-coated microsphere beads incubated with the methylglyoxal, confirming that exposure of the collagen-coated microsphere beads to methylglyoxal did cause modifications of the collagen (i.e., glycation).

### B. Measurement of Glycated Collagen Phagocytosis by HDFs

### Procedure:

1. On day 1, HDFs were plated in 24 well plates.
2. On day 2, HDFs, after reaching confluence, were maintained for 48 hours under starvation conditions.
3. On day 4, HDFs were incubated for 3 hours with collagen-coated microspheres (2×10⁶ beads) as control or with collagen-coated microspheres (2×10⁶ beads) that had been pre-treated overnight with glycation inducer, methylglyoxal. HDFs were then washed with PBS to remove unbound and loosely bound beads.
4. Cell viability was measured using Presto Blue reagent; phagocytosis was measured by the determination of the fluorescence reading of internalized beads, using the procedure described above in Example 1.

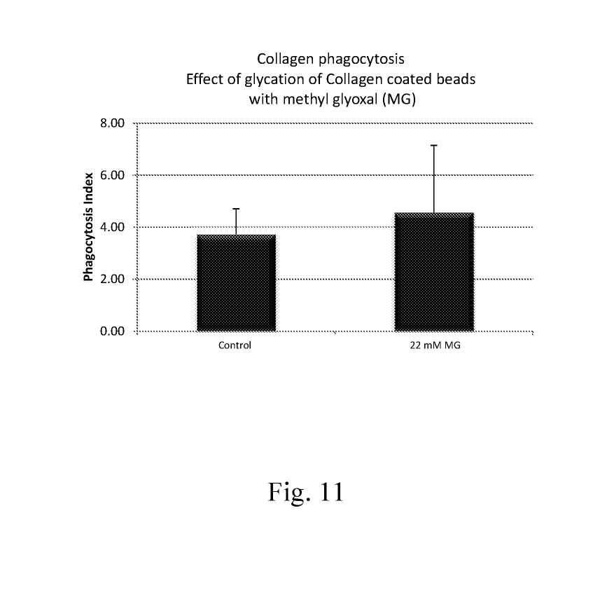

Results, shown in FIG. 11, are expressed as Phagocytosis Index (i.e., the ratio of the phagocytosis fluorescent signal divided by the viability signal). The results indicate that collagen-coated microsphere beads that had been pre-treated with methylglyoxal were taken up by HDFs at substantially the same rate as microsphere beads coated with unmodified collagen. From these results, the inventors theorize that the methods of the invention for boosting collagen turnover could be used successfully to prevent or eliminate signs of aging in difficult to treat areas of the skin, such as the periorbital areas, where collagen is resistant to degradation by MMPs.

While some illustrative embodiments of the invention have been described hereinabove, such illustrative embodiments should not be interpreted in any manner to limit the broad scope of the present invention. Various modifications and equivalents of the described embodiments and components thereof will be apparent to those of ordinary skill in the art. Some modifications and equivalents will be readily recognized by one ordinarily skilled in the art, while others may require no more than routine experimentation. It is therefore understood that such modifications and equivalents are within the spirit and scope of the present invention.

What we claim is:

1. A method for identifying a material having an efficacy for enhancing the activity of a collagen promoting active, comprising the steps of:
(a) incubating human dermal fibroblasts (HDFs) in the presence or absence of a test material;
(b) thereafter, removing the test material and incubating the HDFs in the presence of a collagen-promoting active for a time sufficient to stimulate collagen release by HDFs; and
(c) measuring an amount of collagen released by HDFs treated with the test material and with the collagen promoting active and comparing that amount with an amount of collagen released by HDFs treated with the collagen-promoting active in the absence of the test material.

2. The method of claim 1, wherein in step (a), the HDFs are incubated in the presence of the test material for about 48 hours, and wherein in step (b), the HDFs are incubated in the presence of the collagen promoting active for about 72 hours.

3. The method of claim 1, wherein measuring collagen released in step (c) is carried out using a Procollagen Type 1 C-Peptide Enzyme-linked immunosorbent assay (PIP EIA).

4. The method of claim 1, wherein the collagen synthesis-promoting active is selected from the group consisting of TGFβ1, Solpeptide, Calophyllum Inophyllum (available as Tamanu Original Oil), Palmitoyl Dipeptide-5 diaminobutyroyl hydroxythreonine/Palmitoyl Dipeptide-5 diaminohydroxybutyrate (Syn® Tacks 801045), Mimosa tenuiflora (available as Tepescohuite spray dried extract), Soybean polypetide, Rhodiola Rosea, Soybean extract (available as Phytomatrix K100), Palmitoyl Tripeptide-1/Palmitoyl Tripeptide-7, Algae extract/hydrolyzed Rice protein, Hippophae Rhamnoides (available as Sea Buckthorn extract), Vitis vinifera (available as Neovixyl), Opuntia Tuna (prickly pear cactus), Whey protein (available as NXP75), extract of the yeast Hansenula polymorpha (H. polymorpha), Palmitoyl Pentapeptide-4 (available as Matrixyl 500), Yeast polysaccharides (available as Metabiotics™ Microlift), Laminaria Digitata (available as Mitostime DI), Hydrolyzed collagen, Algae extract (available as Lanablue paraben free), Galactoarabinan (available as TRlspire Enhance), Laurydone, Terminalia Ferdinandiana (Kakadu Plum Extract (Glycerin)), Aminopropyl ascorbyl phosphate, Artemia, Psidium Guajava (Guava Extract), Zea mays, Crithmum Maritimum, Eryngium Maritimum, Creatine (available as Creapure®), Centella asiatica, Persea Gratissima (avocado fruit extract, available as Avocadin™ HU25), Scutellaria Baicalensis (available as Baicalin MM), Palmitoyl hexapeptide-12, Tetrahexyldecyl ascorbate (available as BV-OSC), Pseudoalteromonas ferment (available as Antarcticine®), Acetyl hexapeptide-8 (available as Argireline® solution), ascorbyl glucoside, Magnesium ascorbyl phosphate, and Ascorbyl palmitate, and combinations thereof.

5. A method for identifying a material having an efficacy for enhancing the activity of a collagen synthesis-promoting active, comprising the steps of:
(a) incubating human dermal fibroblasts (HDFs) in the presence of a collagen synthesis- promoting active or in the presence of a combination of a collagen synthesis-promoting active with a test material for a time sufficient to stimulate collagen release by the HDFs; and
(b) measuring an amount of collagen released by HDFs treated with both the test material and the collagen synthesis-promoting active and comparing that amount with an amount of collagen released by HDFs treated with the collagen synthesis-promoting active in the absence of the treatment with the test material.

6. The method of claim 5, wherein the collagen synthesis-promoting active is selected from the group consisting of TGFβ1, Solpeptide, Calophyllum Inophyllum (available as Tamanu Original Oil), Palmitoyl Dipeptide-5 diaminobutyroyl hydroxythreonine/Palmitoyl Dipeptide-5 diaminohydroxybutyrate (Syn® Tacks 801045), Mimosa tenuiflora (available as Tepescohuite spray dried extract), Soybean polypetide, Rhodiola Rosea, Soybean extract (available as Phytomatrix K100), Palmitoyl Tripeptide-1/Palmitoyl Tripeptide-7, Algae extract/hydrolyzed Rice protein, Hippophae Rhamnoides (available as Sea Buckthorn extract), Vitis vinifera (available as Neovixyl), Opuntia Tuna (prickly pear cactus), Whey protein (available as NXP75), extract of the yeast Hansenula polymorpha (H. polymorpha), Palmitoyl Pentapeptide-4 (available as Matrixyl 500), Yeast polysaccharides (available as Metabiotics™ Microlift), Laminaria Digitata (available as Mitostime DI), Hydrolyzed collagen, Algae extract (available as Lanablue paraben free), Galactoarabinan (available as TRlspire Enhance), Laurydone, Terminalia Ferdinandiana (Kakadu Plum Extract (Glycerin)), Aminopropyl ascorbyl phosphate, Artemia, Psidium Guajava (Guava Extract), Zea mays, Crithmum Maritimum, Eryngium Maritimum, Creatine (available as Creapure®), Centella asiatica, Persea Gratissima (avocado fruit extract, available as Avocadin™ HU25), Scutellaria Baicalensis (available as Baicalin MM), Palmitoyl hexapeptide-12, Tetrahexyldecyl ascorbate (available as BV-OSC), Pseudoalteromonas ferment (available as Antarcticine®), Acetyl hexapeptide-8 (available as Argireline® solution), ascorbyl glucoside, Magnesium ascorbyl phosphate, and Ascorbyl palmitate, and combinations thereof.

7. A method of enhancing collagen turnover in the skin, comprising the steps of:
(a) applying to the skin in need of enhanced collagen turnover a first composition comprising a first active having an efficacy for degrading collagen in connective tissue in the skin and a cosmetically acceptable vehicle; and allowing the first composition to remain in contact with the skin for a time sufficient to stimulate collagen phagocytosis by fibroblasts in the connective tissue;
(b) applying to the skin a second composition comprising a second active having an efficacy for promoting collagen synthesis and a cosmetically acceptable vehicle; and allowing the second composition to remain in contact with the skin for a time sufficient to promote collagen release by fibroblasts in the connective tissue in the skin; wherein steps (a) and (b) occur sequentially in any order; and wherein the first active improves the activity of the second active when both the first composition and the second composition are applied to the skin.

8. The method of claim 7, wherein the second active is selected from the group consisting of TGFβ1, Solpeptide, Calophyllum Inophyllum (available as Tamanu Original Oil), Palmitoyl Dipeptide-5 diaminobutyroyl hydroxythreonine/Palmitoyl Dipeptide-5 diaminohydroxybutyrate (available as Syn® Tacks 801045), Mimosa tenuiflora (available as Tepescohuite spray dried extract), Soybean polypetide, Rhodiola Rosea, Soybean extract (available as Phytomatrix K100), Palmitoyl Tripeptide-1/Palmitoyl Tripeptide-7, Algae extract/hydrolyzed Rice protein, Hippophae Rhamnoides (available as Sea Buckthorn extract), Vitis vinifera (available as Neovixyl), Opuntia Tuna (prickly pear cactus), Whey protein (available as NXP75), extract of the yeast Hansenula polymorpha (H. polymorpha), Palmitoyl Pentapeptide-4 (available as Matrixyl 500), Yeast polysaccharides (available as Metabiotics™ Microlift), Laminaria Digitata (available as Mitostime DI), Hydrolyzed collagen, Algae extract (available as Lanablue paraben free), Galactoarabinan (available as TRlspire Enhance), Laurydone, Terminalia Ferdinandiana (Kakadu Plum Extract (Glycerin)), Aminopropyl ascorbyl phosphate, Artemia, Psidium Guajava (Guava Extract), Zea mays, Crithmum Maritimum, Eryngium Maritimum, Creatine (available as Creapure®), Centella asiatica, Persea Gratissima (avocado fruit extract, available as Avocadin™ HU25), Scutellaria Baicalensis (available as Baicalin MM), Palmitoyl hexapeptide-12, Tetrahexyldecyl ascorbate (available as BV-OSC), Pseudoalteromonas ferment (available as Antarcticine®), Acetyl hexapeptide-8 (available as Argireline® solution), ascorbyl glucoside, Magnesium ascorbyl phosphate, and Ascorbyl palmitate, and combinations thereof.

9. The method of claim 7, wherein the first active is selected from the group consisting of Neovixyl, Theobroma, NXP75, Taisoh liquid, Pichia/resveratrol ferment, Wild Bush Plum extract, Amacha liquid B, Bifidus extract, and combinations thereof.

10. The method of claim 7, wherein the skin in need of enhanced collagen turnover is characterized by the presence of stiffened, aligned collagen.

11. The method of claim 10, wherein the skin in need of enhanced collagen turnover is skin of periorbital areas.

12. A method of enhancing collagen turnover in the skin, comprising the steps of:
(a) applying to the skin in need of enhanced collagen turnover a composition comprising a first active having an efficacy for degrading collagen by stimulating collagen phagocytosis by fibroblasts in connective tissue in the skin and a second active having an efficacy for promoting collagen synthesis by fibroblasts in the connective tissue and a cosmetically acceptable vehicle, wherein the first active improves the efficacy of the second active when the composition is applied to the skin; and
(b) permitting the composition to remain in contact with the skin for a time sufficient to enhance collagen turnover in the skin.

13. A skin treatment kit comprising:
a first composition containing a first active having an efficacy for degrading collagen by stimulating collagen phagocytosis by fibroblasts in connective tissue in the skin and a cosmetically acceptable vehicle;
a second composition containing a second active having an efficacy for promoting collagen synthesis by fibroblasts in connective tissue in the skin and a cosmetically acceptable vehicle, wherein the first active improves the efficacy of the second active when both the first composition and the second composition are applied to the skin; and
instructions for consumers on the use of the first and second compositions sequentially in any order to achieve a degree of collagen release which exceeds a degree of collagen release achievable by treatment with the second composition in the absence of treatment with the first composition.

14. The skin treatment kit of claim 13, wherein the second active is selected from the group consisting of Neovixyl, Theobroma, NXP75, Taisoh liquid, Pichia/resveratrol ferment, Wild Bush Plum extract, Amacha liquid B, Bifidus extract, and combinations thereof, and wherein the second composition comprises an active selected from the group consisting of TGFβ1, Solpeptide, Calophyllum Inophyllum (available as Tamanu Original Oil), Palmitoyl Dipeptide-5 diaminobutyroyl hydroxythreonine/Palmitoyl Dipeptide-5 diaminohydroxybutyrate (Syn® Tacks 801045), Mimosa tenuiflora (available as Tepescohuite spray dried extract), Soybean polypetide, Rhodiola Rosea, Soybean extract (available as Phytomatrix K100), Palmitoyl Tripeptide-1/Palmitoyl Tripeptide-7, Algae extract/hydrolyzed Rice protein, Hippophae Rhamnoides (available as Sea Buckthorn extract), Vitis vinifera (available as Neovixyl), Opuntia Tuna (prickly pear cactus), Whey protein (available as NXP75), extract of the yeast Hansenula polymorpha (H. polymorpha), Palmitoyl Pentapeptide-4 (available as Matrixyl 500), Yeast polysaccharides (available as Metabiotics™ Microlift), Laminaria Digitata (available as Mitostime DI), Hydrolyzed collagen, Algae extract (available as Lanablue paraben free), Galactoarabinan (available as TRIspire Enhance), Laurydone, Terminalia Ferdinandiana (Kakadu Plum Extract (Glycerin)), Aminopropyl ascorbyl phosphate, Artemia, Psidium Guajava (Guava Extract), Zea mays, Crithmum Maritimum, Eryngium Maritimum, Creatine (available as Creapure®), Centella asiatica, Persea Gratissima (avocado fruit extract, available as Avocadin™ HU25), Scutellaria Baicalensis (available as Baicalin MM), Palmitoyl hexapeptide-12, Tetrahexyldecyl ascorbate (available as BV-OSC), Pseudoalteromonas ferment (available as Antarcticine®), Acetyl hexapeptide-8 (available as Argireline® solution), ascorbyl glucoside, Magnesium ascorbyl phosphate, and Ascorbyl palmitate, and combinations thereof.

15. The skin treatment kit of claim 13, wherein the first active is selected from the group consisting of Neovixyl, Theobroma, NXP75, Taisoh liquid, Pichia/resveratrol ferment, Wild Bush Plum extract, Amacha liquid B, Bifidus extract, and combinations thereof.

16. A skin treatment product comprising a composition comprising a first active having an efficacy for degrading collagen by stimulating collagen phagocytosis by fibroblasts in connective tissue in the skin and a second active having an efficacy for promoting collagen synthesis by fibroblasts in the connective tissue and a cosmetically acceptable vehicle, wherein the first active and the second active are present in the composition in amounts effective for the first active to improve the efficacy of the second active when the composition is applied to the skin.

17. A method for cosmetically improving human skin through a treatment regime for enhancing collagen turnover in the skin comprising:
providing a first composition containing a first active having an efficacy for degrading collagen in connective tissue in the skin and a cosmetically acceptable vehicle, the first composition functioning to stimulate collagen phagocytosis by fibroblasts in the connective tissue;
providing a second composition containing a second active having an efficacy for promoting collagen synthesis in connective tissue in the skin and a cosmetically acceptable vehicle, the second composition functioning to promote collagen release by fibroblasts in the connective tissue;
instructing consumers on the use of the first and second compositions sequentially to achieve a level of collagen turnover in the skin which exceeds a level of collagen turnover achievable by treatment with the second composition in the absence of treatment with the first composition;
applying the first composition to the skin; and
applying the second composition to the skin; wherein the first composition and the second composition may be applied to the skin in any order; and wherein the first active improves the efficacy of the second active when both the first composition and the second composition are applied to the skin.

18. The method of claim 17, wherein the first active is selected from the group consisting of Neovixyl, Theobroma, NXP75, Taisoh liquid, Pichia/resveratrol ferment, Wild Bush Plum extract, Amacha liquid B, Bifidus extract, and combinations thereof.

19. The method of claim 17, wherein the second is active selected from the group consisting of TGFβ1, Solpeptide, Calophyllum Inophyllum (available as Tamanu Original Oil), Palmitoyl Dipeptide-5 diaminobutyroyl hydroxythreonine/Palmitoyl Dipeptide-5 diaminohydroxybutyrate (available as Syn® Tacks 801045), Mimosa tenuiflora (available as Tepescohuite spray dried extract), Soybean polypetide, Rhodiola Rosea, Soybean extract (available as Phytomatrix K100), Palmitoyl Tripeptide-1/Palmitoyl Tripeptide-7, Algae extract/hydrolyzed Rice protein, Hippophae Rhamnoides (available as Sea Buckthorn extract), Vitis vinifera (available as Neovixyl), Opuntia Tuna (prickly pear cactus), Whey protein (available as NXP75), extract of the yeast Hansenula polymorpha (H. polymorpha), Palmitoyl Pentapeptide-4 (available as Matrixyl 500), Yeast polysaccharides (available as Metabiotics™ Microlift), Laminaria Digitata (available as Mitostime DI), Hydrolyzed collagen, Algae extract (available as Lanablue paraben free), Galactoarabinan (available as TRIspire Enhance), Laurydone, Terminalia Ferdinandiana (Kakadu Plum Extract (Glycerin)), Aminopropyl ascorbyl phosphate, Artemia, Psidium Guajava (Guava Extract), Zea mays, Crithmum Maritimum, Eryngium Maritimum, Creatine (available as Creapure®), Centella asiatica, Persea Gratissima (avocado fruit extract, available as Avocadin™ HU25), Scutellaria Baicalensis (available as Baicalin MM), Palmitoyl hexapeptide-12, Tetrahexyldecyl ascorbate (available as BV-OSC), Pseudoalteromonas ferment (available as Antarcticine®), Acetyl hexapeptide-8 (available as Argireline® solution), ascorbyl glucoside, Magnesium ascorbyl phosphate, and Ascorbyl palmitate, and combinations thereof.

20. A method for cosmetically improving human skin through a treatment for enhancing collagen turnover in the skin, comprising:
(a) providing a composition comprising a first active having an efficacy for degrading collagen in connective tissue in the skin and a second active having an efficacy for promoting collagen synthesis by fibroblasts in the connective tissue and a cosmetically acceptable vehicle; wherein the first active improves the efficacy of the second active when the composition is applied to the skin; and
(b) applying the composition to the skin and retaining the composition in contact with the skin for a time sufficient for the first active to stimulate collagen phagocytosis by the fibroblasts in the connective tissue and for the second active to promote collagen release by the fibroblasts in the connective tissue.
